(19) United States
(12) Patent Application Publication
(10) Pub. No.: US 2017/0258770 A1
(43) Pub. Date: Sep. 14, 2017

(54) Method for Topically Treating Actinic Keratosis on the Full Face or 250 cm2 on the Chest with Ingenol 3-(3,5-diethylisoxazole-4-carboxylate)

(71) Applicant: LEO Laboratories Limited, Dublin (IE)

(72) Inventors: Casper Clemmensen, Ballerup (DK); Johan Selmer, Ballerup (DK); Kim Mark Knudsen, Ballerup (DK)

(21) Appl. No.: 15/529,631

(22) PCT Filed: Nov. 27, 2015
(86) PCT No.: PCT/EP2015/077891

§ 371 Date: May 25, 2017

(30) Foreign Application Priority Data
Nov. 28, 2014 (EP) 14195448.7

(51) Int. Cl. A61K 31/42; A61K 9/06; A61K 9/00

(52) U.S. Cl. CPC A61K 31/42; A61K 9/0014; A61K 9/06

(57) ABSTRACT

The invention relates to the treatment of actinic keratosis on the full face or 250 cm² on the chest with ingenol 3-(3,5-diethylisoxazole-4-carboxylate).

### FIELD OF THE INVENTION

The invention relates to the treatment of actinic keratosis on face/chest with ingenol 3-(3,5-diethylisoxazole-4-carboxylate).

### BACKGROUND

The active compound, ingenol 3-(3,5-diethylisoxazole-4-carboxylate), of the present invention is previously been described in PCT/DK2011/000154. The ingenol 3-(3,5-diethylisoxazole-4-carboxylate) is studied with respect to safety and tolerability for field therapy in 25 cm² on 4 separate areas on the forearm in concentrations of 0.025%, 0.05% and 0.075% in a gel formulation.

Existing topical treatments for actinic keratosis comprises different dosage regimens. All of them extend over weeks and months. Picato® which is launched in many countries around the world for treatment of actinic keratosis has a dosage regimen of two or three days depending on the location of the actinic keratosis and also the concentration of the active compound differs depending on the location of the actinic keratosis. According to the FDA label for Picato®, the size of the treatment area is limited to about 25 cm2 (2 inches×2 inches).

### SUMMARY OF THE INVENTION

The present invention provides a topical treatment regimen with ingenol 3-(3,5-diethylisoxazole-4-carboxylate) for actinic keratosis (AK), which is of short duration and applicable to a large skin area on face/chest. Thus, the present invention provides for a compound different from the active compound, ingenol mebutate, in Picato® in a dosage regimen which is optimized for face/chest. The treatment is simple by the three day regimen. The treatment is directed to include investigating the effect of the treatment on hyperkeratotic/hypertrophic actinic keratosis. The treatment is optimized towards acceptable side-effects in terms of measured local skin reactions (LSR).

The present invention also provides a method of treating a subject diagnosed with actinic keratosis on the face/chest, said method comprising applying an effective amount of active compound to a treatment area for three days to achieve reduction in the number of the actinic keratosis in the treated area.

### DETAILED DESCRIPTION OF THE INVENTION

Actinic Keratosis (AK) is a common skin condition visible as thickened, cornified, more or less scaly lesions, often asymptomatic and characterised histopathologically by proliferation of atypical keratinocytes. It is estimated that AK occurs in 11-50% of the population aged 40 and older in the US and Australia, while the prevalence rate in Europe is 11-25%. Patients with AK tend to have Fitzpatrick skin type I or II (fair skin) which burns with sun exposure and does not tan or tans minimally.

In the context of AK, adjacent AKs may merge into one another producing a field of abnormal skin. Such ‘field cancerisation’ is characterised by the epithelial surface of the photo-damaged area being susceptible to the development of additional AKs or a malignancy. This is evidenced by the presence of multiple subclinical and clinically visible AK lesions as well as multifocal preneoplastic change with genetic mutations. There is also increasing evidence that AK represents squamous cell carcinoma (SCC) in situ in its earliest stages. Histopathological evidence shows that contiguous AK is present in 97% of SCC lesions on sun-damaged skin. AK is linked epidemiologically to development of SCC (14), and both conditions share specific gene expression. If left untreated, AK may progress to SCC, with significant morbidity and death.

Ingenol 3-(3,5-diethylisoxazole-4-carboxylate) is an ingenol analogue, manufactured by a semi-synthetic process from ingenol, and developed and formulated for the field treatment of AK. Ingenol 3-(3,5-diethylisoxazole-4-carboxylate) is in the initial phase of its clinical development program, while ingenol mebutate, another ingenol analogue, has been approved in the USA, EU, Brazil, Australia, and Canada as a field treatment for AK on the face and scalp, trunk and extremities under the brand name of Picato®. The vehicle formulation of ingenol 3-(3,5-diethylisoxazole-4-carboxylate) gel is the same as the vehicle formulation of ingenol mebutate gel. This gel formulation does not contain any inactive ingredients that would result in acute toxicity to the skin and is water soluble.

The exact mechanism of action of ingenol 3-(3,5-diethylisoxazole-4-carboxylate) continues to be investigated, but it is believed that ingenol 3-(3,5-diethylisoxazole-4-carboxylate) has a dual mechanism of action inducing cell death as well as stimulation of immune response. The preclinical safety and tolerability profile of ingenol 3-(3,5-diethylisoxazole-4-carboxylate) is similar to that of ingenol mebutate, but in vivo studies in mice where effects on dermal tumours have been evaluated suggest that ingenol 3-(3,5-diethylisoxazole-4-carboxylate) is more efficacious than ingenol mebutate in eliminating dermal tumours.

It is presently believed that ingenol 3-(3,5-diethylisoxazole-4-carboxylate) has a higher efficacy and/or milder LSR profile than ingenol mebutate.

Ingenol mebutate gel has recently been approved as Picato® for field therapy treatment of AK in the USA, EU, Brazil, Australia, and Canada. Picato® contains the active ingredient ingenol mebutate which is an ingenol analogue. The duration of treatment with ingenol mebutate gel is two to three consecutive days which provides an advantage for treatment compliance and patient convenience. The duration of treatment required for currently marketed topical products ranges from 2 to 16 weeks. It has been previously documented that longer treatment durations reduce patient compliance.

From Picato® and other topical agents used in the treatment of actinic keratosis, it is well known that differences in treatment efficacy exists between different anatomical regions. Regions like scalp, trunk (except chest) and extremities are more difficult to treat than face. From Picato® studies have shown that local skin reactions after a given dose are milder on difficult to treat anatomical regions than on anatomical regions more easily treated with the compound. For Picato® the skin of the chest is treated with a high concentration of compound. However, the skin of the chest is comparable in several parameters to the face. For these reasons the present invention optimizes the treatment of the face/chest only.

The present invention simplifies prior and existing treatments by a simple three day regimen. The present invention describes treatment of actinic keratosis on the face/chest with ingenol 3-(3,5-diethylisoxazole-4-carboxylate).

In the context of the present invention, the treatment area is defined a full face or up to about 250 cm² on the chest.

The term “about” in this context reflects the approximate nature of a treated skin area but should quantified to ±10%. In some embodiments of the invention the term “about” refers to ±10%, in some embodiments the term refers to ±5%, and in other embodiments the term “about” refers to ±2%.

Local skin reactions (LSRs) occur sometimes in the treated area. Often LSRs are quantified by a scale evaluating the following types of skin reactions: erythema, flaking/scaling, crusting, swelling, vasiculation/postulation, and erosion/ulceration which are categorized into categories 0-4 depending on the severity of the reactions.

The present invention utilizes data obtained from a previous dose escalation trial on face/chest, wherein maximal tolerated doses were identified.

In the trial pre-defined grades of LSRs will constitute Dose Limiting Toxicity.

The level of skin reactivity constituting dose limiting toxicity (DLT) does not reflect safety concerns but represent what dermatologists consider limits for peak levels of acceptable visible skin reactions.

Dose Limiting toxicity (DLT) is defined one or more of the following LSRs
crusting Grade 4
Erosion/ulceration Grade 4
vesiculation/postulation Grade 4 or

Two or more of the following five LSRs:
Erythema Grade 4
Crusting Grade 3
Swelling grade 4
Erosion/ulceration Grade 3
vesiculation/postulation Grade 3 or
other clinically relevant signs or symptoms observed, which the Investigator judges to be counted as a DLT.

Maximum tolerated dose (MTD) is declared as dose level at which less than 4 subjects out of experience a DLT of cohort of 12 subjects.

The study is designed in two parts:

Part 1 was a dose escalation study conducted with a once daily application of trial medication for two consecutive days and continued until the MTD was identified.

Dose escalation proceeded if the safety and tolerability data of the subject up to day 8 was reviewed and considered satisfactory. The treatment consisted of once daily treatment for 2 consecutive days. Up to 5 different doses may be investigated in cohorts of 1 subject. The number of subjects in each cohort depended on the number of observed DLTs. However, the MTD was always be confirmed in a cohort of 12 subjects.

For this study MTD was identified at at 0.018%. At this dose LSRs were mild and application site adverse events minimal.

Part 2 is a study of the efficacy of the 0.018% dose identified in part 1 in a three day regimen.

In contrast to other similar trials conducted, this trial allows inclusion in the trial of hypertrophic and hyperkeratotic lesion, and separately, the efficacy for these lesions are evaluated.

The outcome for Part 2 is safety and efficacy in a once daily application for three consecutive days on the face/chest. The efficacy in treating of the AK will be measured as reduction in AK count in the treated area of lesions which are not considered hyperkeratotic/hypertrophic.

In parallel the efficacy in treating of the AK will be measured as reduction in AK count in the treated area of lesions which are considered hyperkeratotic/hypertrophic.

The reduction in AK count will be performed at week 4 and 8 no matter the type of lesion. Also complete clearance of AKs in the treated area which are not considered hyperkeratotic/hypertrophic at week 8 will be measured.

The trial medication applied is preferably a gel. The gel formulation comprises: isopropyl alcohol, hydroxyethyl cellulose, benzyl alcohol, citric acid monohydrate, sodium citrate dehydrate and water and active compound in a 0.018% concentration. For this particular trial, the dosing is two unit dose tubes daily, each containing 0.67 g.

The active compound is ingenol 3-(3,5-diethylisoxazole-4-carboxylate).

The present invention also relates to the treatment of actinic keratosis lesions on face/chest by a once daily, three day treatment.

The invention relates to the treatment of actinic keratosis lesions in an area of the full face or up to approximately 250 cm² on the chest in a three day treatment.

In an embodiment according to the present invention, the three day treatment is three consecutive days.

In another embodiment of the present invention, the present invention provides application of the ingenol 3-(3,5-diethylisoxazole-4-carboxylate) in a concentration of about 0.018%, In another embodiment of the present invention, the present invention provides a method of treating a subject diagnosed with actinic keratosis on the face/chest, said method comprising applying an effective amount of the ingenol 3-(3,5-diethylisoxazole-4-carboxylate) to a treatment area once daily for three consecutive days to achieve reduction in the number of actinic keratosis lesions in the treatment area.

### EXAMPLE 1

To be included in the trial, Part 1, the subjects must be at least 18 years of age.

Subjects who qualify for Part 1 of the trial must have 5 to 20 actinic keratosis on the face or chest. To be included in the trial part 2 the subjects must have 5-20 clinically typical, visible and discrete actinic keratosis on the face or chest

Part 1: Trial medication, ingenol 3-(3,5-diethylisoxazole-4-carboxylate) gel, will be applied to the treatment area once daily for two consecutive days until the maximum tolerated dose has been reached for each formulation. Up to five different doses of active compound in gel may be investigated in cohorts 12 subjects. Predefined grades of Local Skin Responses (LSRs) will constitute dose limiting toxicity (DLT). The maximum tolerated dose is defined as the highest dose level at which less than 4 out of 12 subjects experience a Dose limiting toxicity.

The doses will be administered in an escalating manner following review of safety and tolerability data performed by the dose escalation committee. Evaluation of the safety and tolerability data will be performed after Visit 5/day 8. Then subjects will be followed for 8 weeks after first application of investigational product.

To qualify for part 2 of the trial, the subjects must have 5-20 clinically visible and discrete AKs within full face or 250 cm² area on the chest. In addition to the requirement of 5-20 clinically typical, visible and discrete AKs within the selected treatment area, subjects may also have visible and discrete hyperkeratotic/hypertrophic lesions in this area. The subjects will be treated with a dose of 0.018% as a once daily three consecutive days treatment and followed for 8 weeks.

The trial consists of an initial part, where 18 subjects will be treated on face/chest. The selected dose was well tolerated both with respect to LSRs and application site reaction in the two days dosing regimen. As this is a safety trial, it is considered justified to conduct this clinical trial as an open-label, uncontrolled trial. An early safety and tolerability evaluation will be performed following the 3 days treatment. If safety and tolerability is found acceptable by the Early Data Review Committee, and additional 44 subjects will be enrolled in order to expand safety observations and obtain an estimate of treatment efficacy.

Another aspect of the trial will be to investigate the effect of the compound on hyperkeratotic/hypertrophic lesions. The treatment effect towards these lesions is not well known, and the lesions will be marked and followed separately, ensuring that treatment efficacy of the compound against clinically typical visible and discrete AKs can be followed as normal.

Exclusion criteria is incompletely healed wound, basal cell carcinoma or squamous cell carcinoma within 5 cm of treatment area, or prior treatment with ingenol mebutate gel on the treatment area, or atypical clinical appearance on the lesions such as cutaneous horns, and/or recalcitrant disease such as non-responding to cryotherapy on two previous occasions. Also other skin conditions, cosmetic procedures or other disease or medication which could interfere with the evaluation of the trial medication or the assessments of the treated area are exclusion criteria, as is other disease or medical conditions which make the subject unsuitable to participate in the trial.

The patients are scheduled for 7 visits:
Visit 1: within 21 days prior to day 1
Visit 2: day 1 (application of trial medication)
Visit 3: day 4 (application of trial medication)
Visit 4: day 8 (±2 day)
Visit 5: week 2 (±2 day)
Visit 6: week 4 (±4 day)
Visit 7: week 8 (±7 day)

LSRs are evaluated at all visits following visit 1.

LSRs are evaluated at all visits following visit 1. AK lesion counts are performed on Visit 2, 6 and 7.

Efficacy analyses will be based on the full analysis set, which will be defined as all randomised subjects. Per protocol analysis set will be used as an efficacy subset and will be defined as subjects in the full analysis set who complete the study without major protocol deviations. Excluding subjects who receive no treatment with the investigational product, provide no efficacy data following start of treatment, have taken the wrong IP or do not fulfill the disease defining inclusion criteria. Further exclusion of subjects of data will be decided upon after a review of the data reviewing all the remaning in- and exclusion criteria, but focusing on concomitant medication that may affect actinic keratosis and also considering compliance/adherence and violations of visit windows.

The number of subjects experiencing DLTs based on LSRs up to and including day 8 will be tabulated by treatment group for the safety analysis set.

The following will also be assessed during the trial:

Assessment by Investigator:

At Baseline (Day 1) and Visit 7 (Week 8), the investigator will make a clinical (visual and tactile) assessment of the extent of photo-damage in the treatment area with respect to 1) fine wrinkling, 2) coarse wrinkling, 3) mottled pigmentation, 4) roughness, 5) sallowness, 6) skin laxity, and 7) telangiectasia. Severity for each of these characteristics will be on a 5-point scale: None (0), Mild (1), Moderate (2), Severe (3), Extreme (4).

At Visit 7 (Week 8) the Investigator will make an overall clinical (visual and tactile) assessment of the subject's photo-damage change from baseline in the treatment area (including an integrated assessment of fine wrinkling, coarse wrinkling, mottled pigmentation, roughness, sallowness, skin laxity, and telangiectasia based on the subject's appearance at the baseline visit).

The scoring will be on a 7-point symmetric scale: Marked improvement (+3), Moderate improvement (+2), Minor improvement (+1), No change (0), Minor worsening (−1), Moderate worsening (−2), Marked worsening (−3).

Patient Reported Outcomes:

The subject must make self-assessments at visits specified in the schedule of trial procedures. Patient reported outcome measures should be completed prior to other assessments on the day of completion of the questionnaire. The subjects should be encouraged to answer all questions in the questionnaire.

Treatment Satisfaction Questionnaire for Medication (TSQM):

TSQM is a generic questionnaire measuring subjects' satisfaction with the treatment. The questionnaire will ask questions relating to effectiveness, side effects, convenience and overall satisfaction. If a subject withdraws from or completes the trial prior to Visit 7 (Week 8), the TSQM questionnaire should be completed at the time of withdrawal/completion in lieu of Visit 7 (Week 8).

Work Productivity and Activity Impairment (WPAI):

At visits specified in the schedule of trial procedures the subjects will complete a self-assessment questionnaire evaluating the effect of the underlying disease on the subject's ability to work and perform regular activities. The WPAI is a widely used questionnaire to capture productivity loss.

Cosmetic Outcome Assessment by Subject:

At Visit 7 (Week 8), the subjects will complete a self-assessment questionnaire evaluating the change in the 1) overall appearance of the skin and 2) overall feel of the skin after treatment. The scoring will be on a 5-point scale: Much worsened, Somewhat worsened, No change, somewhat improved, much improved.

The secondary response criteria will be analysed for the full analysis set and for the per protocol analysis set. The ratio of number of AK lesions at week 8 relative to baseline, excluding lesions identified at base line as hyperkeratotic/hypertrophic lesions, will be tabulated by treatment group. Separately for each treatment group, the rate and corresponding 95% confidence interval will be estimated from a negative binomial regression on the AK count at week 8 with the log baseline value as an offset variable.

The same analysis will be performed for the subgroup of lesions identified as hyperkeratotic/hypertrophic at baseline.

The same analyses will be conducted for the number of AK lesions at week 4.

The number and percentage of subjects with complete clearance at week 8 excluding lesions identified at baseline as hyperkeratotic/hypertrophic lesions, will be tabulated by treatment group with an exact 95% CI.

Results

In the present open label study 63 patients were included and 95% completed the study. The treatment was effective with a reduction in AK count of 81% and a complete clearance of 37% at week 4 and the same in week 8.

The side-effects were considered acceptable. They maximize at day 4, with a rapid decline thereafter. The cosmetic outcome of the treatment was good, with photo damage improved in two-thirds of patients, and with improvement in appearance and feel reported by more than 90% of subjects.

### CONCLUSION

The above invention provides an effective, tolerable treatment of AK in full face or up to 250 cm² of the chest with a high compliance and good cosmetic outcome.

1. A method of treating a subject diagnosed with actinic keratosis on the face and/or chest, said method comprising applying an effective amount of ingenol 3-(3,5-diethylisoxazole-4-carboxylate) to a treatment area on the full face or 250 cm² on the chest for three days.

2. The method of claim 1, wherein the method provides a reduction in the number of actinic keratosis lesions in the treatment area.

3. The method of claim 1, wherein the three day treatment is three consecutive days.

4. The method of claim 1, wherein the treatment area is up to about 250 cm².

5. The method of claim 1, wherein the effective amount of the ingenol 3-(3,5-diethylisoxazole-4-carboxylate) is applied from a dosage strength formulation of ingenol 3-(3,5-diethylisoxazole-4-carboxylate) of about 0.018%

6. The method of claim 1, wherein the amount of the ingenol 3-(3,5-diethylisoxazole-4-carboxylate) applied is about 0.9 mg ingenol 3-(3,5-diethylisoxazole-4-carboxylate)/per day/250 cm² treatment area.

7. The method of claim 1, wherein the ingenol 3-(3,5-diethylisoxazole-4-carboxylate) is formulated in a gel.

8. The method of claim 1, wherein the ingenol 3-(3,5-diethylisoxazole-4-carboxylate) is topically applied in a concentration of about 0.018%.

9. A method of treating a subject diagnosed with actinic keratosis on the face and/or chest, said method comprising topically applying an effective amount of ingenol 3-(3,5-diethylisoxazole-4-carboxylate) to a treatment area on the full face or 250 cm² on the chest for three consecutive days, wherein said method provides a reduction in the number of actinic keratosis lesions in the treated area on the face/chest, wherein the treatment area of the chest is of a size up to about 250 cm², and wherein the dosage strength of the ingenol 3-(3,5-diethylisoxazole-4-carboxylate) is about 0.018%.
